(19) United States
(12) Patent Application Publication
(10) Pub. No.: US 2009/0082386 A1
(43) Pub. Date: Mar. 26, 2009

(54) Ascomycin and pimecrolimus having reduced levels of desmethylascomycin and 32-deoxy-32-epichloro-desmethylascomycin respectively, and methods for preparation thereof

(76) Inventors: Erzsebet Meszarosne Sos, Debrecen (HU); Vilmos Keri, Debrecen (HU); Andrea Csorvasi, Debrecen (HU); Viktor Gyollai, Tiszafured (HU); Piroska Kovacs, Debrecen (HU); Angela Simon, Debrecen (HU); Jozsef Simulak, Hajdusamson-Samsonkert (HU)

Correspondence Address: KENYON & KENYON LLP, ONE BROADWAY, NEW YORK, NY, 10004, US

(21) Appl. No.: 11/983,124

(22) Filed: Nov. 6, 2007

Related U.S. Application Data
(60) Provisional application No. 60/857,419, filed on Nov. 6, 2006.
(60) Provisional application No. 60/962,633, filed on Jul. 30, 2007.
(60) Provisional application No. 60/998,770, filed on Oct. 11, 2007.

(51) Int. Cl. A61K 31/436; C07D 267/00; A61P 17/00

(52) U.S. Cl. 514/291; 540/456

(57) ABSTRACT

Provided is ascomycin that has a low level of an FK523 impurity, and pimecrolimus that has a low level of a 32-deoxy-32-epichloro-FK523 impurity, methods of preparing them, and the use of such pimecrolimus for preparing a pharmaceutical composition.

### CROSS REFERENCE TO RELATED APPLICATIONS

The present application claims the benefit of the following U.S. Provisional Patent Application Nos. : 60/857,419, filed Nov. 6, 2006; 60/962,633, filed Jul. 30, 2007; and 60/998,770 filed Oct. 11, 2007. The contents of these applications are incorporated herein by reference.

### FIELD OF THE INVENTION

The present invention relates to ascomycin that has a low level of an FK523 impurity, and pimecrolimus that has a low level of a 32-deoxy-32-epichloro-FK523 impurity, methods of preparing them, and the use of such pimecrolimus for preparing a pharmaceutical composition.

### BACKGROUND OF THE INVENTION

Pimecrolimus, (1R,9S,12S,13R,14S,17R,18E,21 S,23S,24R,25S,27R)-12-[(1E)-2-{(1R,3R,4S)-4-chloro-3-methoxycyclohexyl}-1-methylvinyl]-17-ethyl-1,14-dihydroxy-23,25-dimethoxy-13,19,21,27-tetramethyl-11,28-dioxa-4-aza-tricyclo[22.3.1.0^{4,9}]octacos-18-ene-2,3,10,16-tetraone of the following formula:

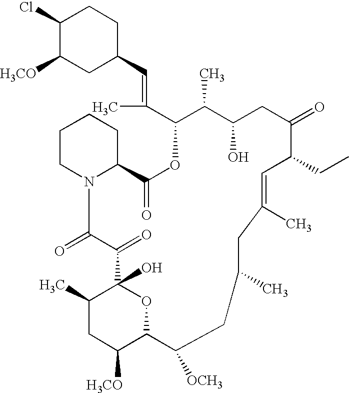

is an anti-inflammatory compound derived from the macrolactam natural product ascomycin of the following formula:

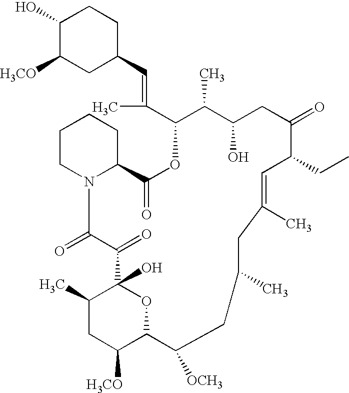

Pimecrolimus is the 32-epichloro derivative of ascomycin, produced by certain strains of Streptomyces.

Pimecrolimus is sold in the United States under the brand name ELIDEL®, and is approved for the treatment of atopic dermatitis.

European Patent EP 427 680 B1 discloses a method of synthesizing pimecrolimus from ascomycin, the only starting material for pimecrolimus known from the literature. The synthesis of ascomycin is by fermentation.

One of the known impurities of the fermentation process is a lower homolog FK523, desmethyl ascomycin:

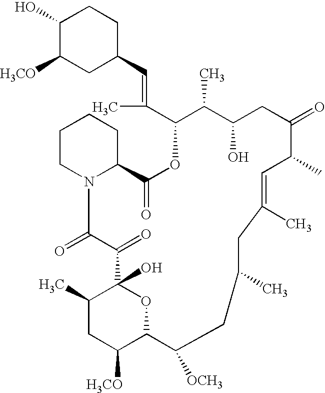

WO patent application discloses that the lower homolog is usually present in the range of 4.7 to 18% area by HPLC and that it presence in Ascomycin is undesirable as it has decreased immunosuppressive activity than ascomycin. It also discloses that the separation of FK-523 from Ascomycin is difficult since it differs from ascomycin in that only one substituent is altered (there is a methyl group instead of an ethyl group at position C-21), and thus having almost the same physical properties such as solubility.

During the conversion of ascomycin to pimecrolimus, the FK-523 impurity is chlorinated providing a new impurity, 32-deoxy-32-epichloro-FK523 of the following formula:

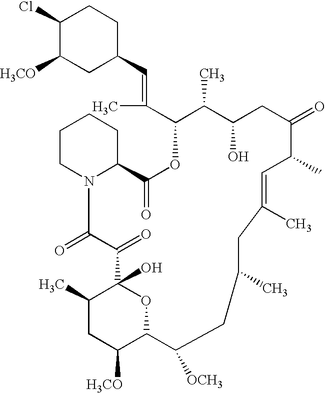

Once again, the chlorinated derivative of FK 523 differs from pimecrolimus in only one group, a methyl group instead of an ethyl group at position C-21. Hence, the removal of this impurity presents a difficult purification problem to the producer of this pharmaceutical.

The existing methods for purifying such macrolides are disclosed in several publications.

U.S. Pat. No. 6,423,722 discloses crystalline macrolides and methods for preparing them.

U.S. Pat. No. 7,220,357 discloses purification of macrolides by column chromatography.

U.S. Pat. No. 3,244,592 reports crystallization of ascomycin by dissolving it in ether and adding hexane to precipitate it.

U.S. Pat. No. 4,894,366 describes purification of ascomycin by dissolving it in ether and precipitating over-night, recovering the product and recrystallizing it from ether.

US patent application No. 2006/0155119 describes crystalline forms of ascomycin, and their preparation.

Hantanaka et al. J. Antibiotics 41, 1592-1601, (1988), Biotechnology and Bioengineering 59, 595-604, (1998) disclose methods for producing ascomycin and purifying it by extraction and crystallization.

US patent application No. 2006/0142564 and US patent application No. 2006/0135548 report about pimecrolimus having a purity of at least 95% area by HPLC and about methods for preparation thereof.

Like any synthetic compound, Pimecrolimus can contain extraneous compounds or impurities, such as 32-deoxy-32-epichloro-FK523. Impurities in Pimecrolimus, or any active pharmaceutical ingredient (“API”), are undesirable and, in extreme cases, might even be harmful to a patient being treated with a dosage form containing the API.

The purity of an API produced in a manufacturing process is critical for commercialization. The U.S. Food and Drug Administration (“FDA”) requires that process impurities be maintained below set limits. For example, in its ICH Q7A guidance for API manufacturers, the FDA specifies the quality of raw materials that may be used, as well as acceptable process conditions, such as temperature, pressure, time, and stoichiometric ratios, including purification steps, such as crystallization, distillation, and liquid-liquid extraction. See ICH Good Manufacturing Practice Guide for Active Pharmaceutical Ingredients, Q7A, Current Step 4 Version (Nov. 10, 2000).

The product of a chemical reaction is rarely a single compound with sufficient purity to comply with pharmaceutical standards. Side products and by-products of the reaction and adjunct reagents used in the reaction will, in most cases, also be present in the product. At certain stages during processing of an API, such as pimecrolimus, it must be analyzed for purity, typically, by high performance liquid chromatography (“HPLC”) or thin-layer chromatography (“TLC”), to determine if it is suitable for continued processing and, ultimately, for use in a pharmaceutical product. The FDA requires that an API is as free of impurities as possible, so that it is as safe as possible for clinical use. For example, the FDA recommends that the amounts of some impurities be limited to less than 0.1 percent. See ICH Good Manufacturing Practice Guide for Active Pharmaceutical Ingredients, Q7A, Current Step 4 Version (Nov. 10, 2000).

Generally, side products, by-products, and adjunct reagents (collectively “impurities”) are identified spectroscopically and/or with another physical method, and then associated with a peak position, such as that in a chromatogram, or a spot on a TLC plate. See Strobel, H. A., et al., CHEMICAL INSTRUMENTATION: A SYSTEMATIC APPROACH, 953, 3d ed. (Wiley & Sons, New York 1989). Once a particular impurity has been associated with a peak position, the impurity can be identified in a sample by its relative position in the chromatogram, where the position in the chromatogram is measured in minutes between injection of the sample on the column and elution of the impurity through the detector. The relative position in the chromatogram is known as the “retention time.”

As is known by those skilled in the art, the management of process impurities is greatly enhanced by understanding their chemical structures and synthetic pathways, and by identifying the parameters that influence the amount of impurities in the final product.

Thus, providing ascomycin having reduced levels of FK523 and methods for preparation thereof would be advantageous. Likewise, providing pimecrolimus having reduced levels of 32-deoxy-32-epichloro-FK523 and methods for preparation thereof would be advantageous.

### SUMMARY OF THE INVENTION

In one embodiment, the present invention provides ascomycin that has a purity of at least about 99.2% area.

In another embodiment, the present invention provides ascomycin with less than about 0.36% area of FK523.

In yet another aspect, the present invention provides ascomycin having at least one of the following quality parameters: a purity of at least about 99.2%, less than about 0.36% area of FK523, and combination thereof.

In one embodiment, the present invention provides a method for obtaining the above ascomycin by a process comprising providing a preliminary purified ascomycin and crystallizing it from a mixture of methanol as a solvent and water as an anti-solvent at a temperature of at least about 45° C., more preferably at least about 60° C.

In another embodiment, the present invention provides a method for crystallizing ascomycin from a mixture of an alcohol as a solvent and water as an anti-solvent at a temperature of at least about 45° C., more preferably at least about 60° C.

In yet another aspect, the present invention provides a process for preparing pimecrolimus comprising preparing ascomycin according to the process of the present invention, and converting it to pimecrolimus; wherein ascomycin has at least one of the following quality parameters: a purity of at least about 99.2% area, less than about 0.36% area of FK523, and combination thereof. Preferably, the obtained pimecrolimus has less than about 0.45% area of 32-deoxy-32-epichloro-FK523.

In one embodiment, the present invention provides pimecrolimus with less than about 0.45% area of 32-deoxy-32-epichloro-FK523. Preferably, pimecrolimus also has a purity of at least about 99.4% area.

In yet another aspect, the present invention provides a process for preparing pimecrolimus with less than about 0.45% area of 32-deoxy-32-epichloro-FK523 comprising a) measuring the purity of the ascomycin in at least one batch of ascomycin; b) selecting a batch of ascomycin having less than about 0.36% area of FK523, and c) preparing pimecrolimus with less than about 0.45% area of 32-deoxy-32-epichloro-FK523 from the selected batch. Preferably, pimecrolimus also has a purity of at least about 99.4% area.

In one embodiment, the present invention provides pharmaceutical formulations comprising the above pimecrolimus and a pharmaceutically acceptable excipient.

In another embodiment, the present invention provides a process for preparing pharmaceutical formulations comprising the above pimecrolimus and a pharmaceutically acceptable excipient.

In yet another aspect, the present invention provides a method for treating a patient suffering from atopic dermatitis, comprising the step of administering to the patient the pharmaceutical formulation of the above pimecrolimus.

In yet another aspect, the present invention provides the use of the above pimecrolimus for the manufacture of a medicament for the treatment of a patient suffering from atopic dermatitis.

### DETAILED DESCRIPTION OF THE INVENTION

The present invention provides ascomycin and pimecrolimus containing a reduced level of the FK523 and 32-deoxy-32-epichloro-FK523 impurities, respectively.

Since the chlorinated impurity in pimecrolimus is structurally related to pimecrolimus, it is difficult to separate it from pimecrolimus using conventional purification methods (see example 3). However, the non chlorinated impurity, i.e., FK-523 can be removed quite easily and efficiently from ascomycin, thus providing a high quality ascomycin that can be used to prepare a high quality pimecrolimus.

The present invention provides ascomycin that has a purity of at least about 99.2% area, more preferably of at least about 99.5% area. Typically, the purity is measured by an HPLC method.

Preferably, the HPLC method used to measure the purity of ascomycin comprises:
a) combining a sample comprising of ascomycin with acetonitrile to obtain a solution;
b) injecting the solution to a an octahedral silane (OSD or C18) chemically bonded to silica gel based HPLC column;
c) eluting the sample from the column using a gradient eluent of a mixture of acetonitrile, water, and acetic acid, referred to as mobile phase A, and a mixture of acetonitrile and acetic acid, referred to as mobile phase B, and
d) measuring the purity of ascomycin using a UV detector.

The present invention also provides ascomycin with less than 0.36% area of FK523, more preferably, with less than 0.2% area of FK523, most preferably, with about 0.15% area to about 0.2% area of FK-523. Typically, the level of FK523 is measured by an HPLC method. Preferably, the HPLC method is the one provided for measuring the purity of ascomycin.

In addition, the present invention provides ascomycin having at least one of the following quality parameters: a purity of at least about 99.2% area, less than 0.36% area of FK523, and combination thereof.

The above ascomycin can be obtained by a method comprising providing a preliminary purified ascomycin, and crystallizing the ascomycin from a mixture of an alcohol as a solvent and water as an anti-solvent at a temperature of at least about 45° C. more preferably at least about 60° C.

As used here the term “preliminary purified” refers to ascomycin that is partially purified by a process comprising column chromatography and crystallization or only by column chromatography. Such preliminary purified ascomycin can be obtained for example, by performing the chromatography process disclosed in EP patent publication No. 1558622 or by combining the chromatography with the crystallization process disclosed in U.S. Pat. No. 7,232,486. EP patent publication No. 1558622 and U.S. Pat. No. 7,232,486 are incorporated herein by reference in their entirety for their teaching of preparing a “preliminary purified” ascomycin.

Generally, the crystallization to obtain a “preliminary purified” ascomycin can be carried out by combining ascomycin with a suitable solvent, such as a C₅-C₇ ester, C₄-C₈ saturated hydrocarbon, or a mixture thereof, and adding water to precipitate the ascomycin. Preferred solvents are ethyl acetate and hexane. In one embodiment of U.S. Pat. No. 7,232,486, the crystallization can be carried out by combining, preferably at a temperature of about 20° C. to about 25° C., ascomycin, ethyl acetate, n-hexane, and a water solution of a base selected from NaOH, KOH, Ca(OH)₂, NH₃, (C₂H₅)₃N, diethylamine and pyridine whereby at least two phases are formed, one of which is a water-rich phase, wherein the pH of the water-rich phase is >about 7, b) maintaining the combination, preferably at a temperature of about 20° C. to about 25° C. for at least 1 hour, whereby an ascomycin rich phase is formed from which ascomycin crystallizes, c) maintaining the combination, preferably at a temperature of about 0° C. to about 20° C. for at least 1 hour, and d) recovering the ascomycin.

Chromatography to obtain a “preliminary purified” ascomycin can be carried out with a suitable resin, such as a polystyrene-divinyl benzene copolymer resin. The ascomycin is dissolved in a suitable solvent such as acetone and combined with the resin and water. A mixture of tetrahydrofuran and water can be used to elute the ascomycin from the resin. Phosphoric acid can be added to prevent decomposition of ascomycin. The ascomycin can then be extracted into a water immiscible organic solvent, such as C₄-C₈ esters, C₁-C₈ chlorinated hydrocarbons, C₃-C₈ ketones. Preferably ethyl acetate is used. Ammonia or another basic agent can be added to facilitate the extraction. The ascomycin can then be recovered as a residue by removing the ethyl acetate, such as by evaporation under a pressure of less than one atmosphere.

The recovered ascomycin from chromatography can be crystallized. Crystallization can be carried out by combining the ascomycin with ethyl acetate and optionally a C₅-C₈ saturated hydrocarbon, such as hexane. Water is then added to precipitate the ascomycin.

The preliminary purified ascomycin is then crystallized from a mixture of an alcohol as a solvent and water as an anti-solvent at a temperature of at least about 45° C., more preferably at least about 60° C. according to the process of the present invention.

Typically, the crystallization comprises, dissolving the preliminary purified ascomycin in an alcohol and adding water to the solution to obtain a suspension comprising of precipitated ascomycin; wherein the dissolution and precipitation are done at a temperature of at least about 60° C. Performing the precipitation at a temperature of at least about 60° C., makes the precipitation process more selective.

Preferably, the alcohol is a C₁₋₄ alcohol, more preferably, methanol, ethanol, isopropyl alcohol, n-propyl alcohol, or n-butyl alcohol. Most preferably, the alcohol is methanol.

Preferably, the addition of water is done drop-wise. Preferably, the drop-wise addition is done over a period of about 10 to about 600 minutes, more preferably, over a period of about 1 to about 5 hours.

Typically, the yield of the precipitated ascomycin can be increased by maintaining the suspension for a period of about 0 to about 10 hours, more preferably, for about 1 to about 3 hours, most preferably, for about 2 hours.

Typically, the precipitated ascomycin is then recovered from the suspension. The recovery can be done by filtration. The recovered ascomycin, preferably by filtration, can be dried at a temperature of about 30 to 80° C., more preferably at about 40° C. to 70° C.

The above crystallization can be repeated several times if needed, in order to increase the purity of ascomycin.

The above ascomycin, i.e, ascomycin having at least one of the following quality parameters: a purity of at least about 99.2% area, less than about 0.36% area of FK523, and combination thereof is then used to prepare pimecrolimus. Preferably, the obtained pimecrolimus has less than about 0.45% area of 32-deoxy-32-epichloro-FK523. More preferably, the obtained pimecrolimus has also a purity of at least about 99.4% area.

The process for preparing pimecrolimus with less than about 0.45% area of 32-deoxy-32-epichloro-FK523 comprises a) measuring the purity of the ascomycin in at least one batch of ascomycin; b) selecting a batch of ascomycin having less than about 0.36% area of FK523, and c) preparing pimecrolimus with less than about 0.45% area of 32-deoxy-32-epichloro-FK523 from the selected batch. Preferably, pimecrolimus also has a purity of at least about 99.4% area. Typically, the purity is measured by an HPLC method.

Preferably, the HPLC method used to measure the purity of pimecrolimus comprises:
a) combining a sample comprising of ascomycin with acetonitrile to obtain a solution;
b) injecting the solution to a an octahedral silane (OSD or C18) chemically bonded to silica gel based HPLC column;
c) eluting the sample from the column using a gradient eluent of a mixture of acetonitrile and an aqueous solution of KH₂PO₄ in a ratio of about 75:25, respectively, referred to as mobile phase A, and a mixture of acetonitrile and an aqueous solution of KH₂PO₄ in a ratio of about 80:20, referred to as mobile phase B, and
d) measuring the purity of pimecrolimus using a UV detector.

The conversion of the ascomycin of the present invention to pimecrolimus can be done, for example, according to the process disclosed in US patent application No. 20060142564, incorporated herein by reference. The process of US patent application No. 20060142564 comprises: a) dissolving ascomycin in an organic solvent; b) combining ascomycin with a base and a conversion reagent to obtain an activated ascomycin derivative; c) reacting the activated derivative of ascomycin with a chloride ion source to obtain pimecrolimus; and d) recovering the obtained pimecrolimus. Examples of organic solvents are dichloromethane, chloroform, diethylether, diisopropylether, methyl-t-butylether, toluene, ethyl acetate, i-butylacetate, acetone, methylethylketone, acetonitrile, N,N-dimethylformamide, N,N-dimethylacetamide, dimethylsulfoxide, and mixtures thereof. Examples of activated ascomycin derivatives are sulfonate esters, tosylates or mesylates and triflates. Examples of bases are triethylamine, diisopropyl-ethylamine (EDIPA), N-methyl-morpholine, N,N-dimethylaniline, pyridine, and substituted pyridine derivatives, such as 2,6-lutidine, s-collidine, and 4-dimethylaminopyridine. Examples of conversion reagents are fluorosulfonic anhydride, fluorosulfonyl chloride, trifluoromethanesulfonic anhydride, trifluoromethanesulfonyl chloride, methanesulfonic anhydride, methanesulfonyl chloride, phenylmethanesulfonic anhydride, phenylmethanesulfonyl chloride, p-toluenesulfonic anhydride, p-toluenesulfonyl chloride, benzenesulfonic anhydride, and benzenesulfonyl chloride

Thus, the present invention also provides pimecrolimus that has less than about 0.45% area of 32-deoxy-32-epichloro-FK523. Preferably, pimecrolimus also has a purity of at least about 99.4% area as area percent HPLC.

The present invention provides pharmaceutical formulations comprising the above pimecrolimus and a pharmaceutically acceptable excipient.

The quality of ascomycin, typically affect the quality of the pimecrolimus that is obtained from it, i.e., the level of FK-523 that is present in ascomycin is similar to the level of its chlorinated analogue that contaminates pimecrolimus, as exemplified in example 5.

One aspect of the present invention provides a process for preparing pharmaceutical formulations comprising the above pimecrolimus and a pharmaceutically acceptable excipient.

Another aspect of the present invention provides a method for treating a patient suffering from atopic dermatitis, comprising the step of administering to the patient the pharmaceutical formulation of the above pimecrolimus.

In yet another aspect, the present invention provides the use of the above pimecrolimus for the manufacture of a medicament for the treatment of a patient suffering from atopic dermatitis.

“Therapeutically effective amount” means the amount of the purified pimecrolimus, when administered to a patient for treating a disease or other undesirable medical condition, is sufficient to have a beneficial effect with respect to that disease or condition. The “therapeutically effective amount” will vary depending on the purity, the disease or condition and its severity, and the age, weight, etc. of the patient to be treated. Determining the therapeutically effective amount of a given pure pimecrolimus is within the ordinary skill of the art, and requires no more than routine experimentation.

Pharmaceutical formulations of the present invention contain the purified Pimecrolimus produced by the processes of the present invention. In addition to the active ingredient(s), the pharmaceutical formulations of the present invention may contain one or more excipients. Excipients are added to the formulation for a variety of purposes.

Diluents may be added to the formulations of a present invention. Diluents increase the bulk of a solid pharmaceutical composition, and may make a pharmaceutical dosage for containing the composition easier for the patient and caregiver to handle. Diluents for solid compositions include, for example, microcrystalline cellulose (e.g., AVICEL®, microfine cellulose, lactose, starch, pregelatinized starch, calcium carbonate, calcium sulfate, sugar, dextrates, dextrin, dextrose, dibasic calcium phosphate, dehydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

Solid pharmaceutical compositions that are compacted into dosage form, such as a tablet, may include excipients whose functions include helping to bind the active ingredient and other excipients together after compression. Binders for solid pharmaceutical compositions include acacia, alginic acid, carbomer (e.g., carbopol), carboxymethylcellulose sodium, dextrin, ethyl cellulose, gelatine, guar gum, hydrogenated vegetable oil, hydroxyethyl cellulose, hydroxypropyl cellulose (e.g., KLUCEL®), hydroxypropyl methyl cellulose (e.g., METHOCEL®), liquid glucose, magnesium aluminium silicate, maltodextrin, methylcellulose, polymethacrylates, povidone (e.g., KOLLIDON® PALSDONE®), pregelatinized starch, sodium alginate, and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g., AC-DI-SOL®, PRIMELOSE®), colloidal silicon dioxide, croscarmellose sodium, crospovidone (e.g., KOLLIDON®, POLYPLASDONE®), guar gum, magnesium aluminium silicate, methyl cellulose, microcrystalline cellulose, polacrilin potassium, powdered cellulose, pregelatinized starch, sodium alginate, sodium starch glycolate (e.g., EXPLOTAB®), and starch.

Glidants can be added to improve the flowability of a non-compacted solid composition, and to improve the accuracy of dosing. Excipients that may function as glidants include colloidal silicon dioxide, magnesium trisilicate, powdered cellulose, starch, talc, and tribasic calcium phosphate.

When a dosage form such as tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion, and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavouring agents and flavour enhancers make the dosage form more palatable to the patient. Common flavouring agents and flavour enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance, and/or facilitate patient identification of the product and unit dosage level.

In liquid pharmaceutical compositions prepared using purified Pimecrolimus produced by the processes of the present invention, Pimecrolimus and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatine guar gum, hydroxyethyl cellulose, hydroxypropyl cellulose, hydroxpropyl methyl cellulose, maltodextrin, polyvinyl alcohol, povidone, propylene carbonate, propylene glycol alginate, sodium alginate, sodium starch glycolate, starch tragacanth, and xantham gum.

Sweetening agents such as sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated, hydroxyanisole, and ethylenediamine tetraacetic acid may be added at levels safe for ingestion to improve storage stability.

A liquid composition may also contain a buffer such as gluconic acid, lactic acid, citric acid or acetic acid, sodium gluconate, sodium lactate, sodium citrate, or sodium acetate. Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant, and ophthalmic, administration. Although the most suitable administration in any given case will depend on the nature and severity of the condition being treated, the most preferred route of the present invention is oral.

The dosages may be conveniently presented in unit dosage form, and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The oral dosage form of the present invention is preferably in the form of an oral capsule having a dosage of about 10 mg to about 160 mg, more preferably from about 20 mg to about 80 mg, and most preferably capsules of 20, 40, 60, and 80 mg. Daily dosage may include 1, 2, or more capsules per day.

The dosage form of the present invention may be a capsule containing the composition, preferably a powdered or granulated solid composition of the invention, within either a hard or soft shell. The shell may be made from gelatin, and, optionally, contain a plasticizer such as glycerine and sorbitol, and an opacifying agent or colorant.

A composition for tableting or capsule filling may be prepared by wet granulation. In wet granulation, some or all of the active ingredients and excipients in powder form are blended, and then further mixed in the presence of a liquid, typically water, that causes the powders to clump into granules. The granulate is screened and/or milled, dried, and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet, and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules.

Excipients that are particularly well suited for direct compression tableting include microcrystalline cellulose, spray dried lactose, dicalcium phosphate dihydrate, and colloidal silica. The proper use of these and other excipients in direct compression tableting is know to those in the art with experience and skill in particular formulation challenges of direct compression tableting.

A capsule filling of the present invention may comprise any of the aforementioned blends and granulates that were described with reference to tableting, however, they are not subjected to a final tableting step.

The active ingredient and excipients may be formulated into compositions and dosage forms according to methods know in the art.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### HPLC Method for Analyzing Ascomycin

| Column & | Octadecyl silane (OSD or C18) chemically bonded to | | |
|---|---|---|---|
| Packing: | porous silica particles;; 150 × 4.6 mm, 3.5 μm | | |
| Eluent A: | 200 mL of acetonitrile in 2000 mL of water and 100 μL of | | |
| | 50% solution of acetic acid | | |
| Eluent B: | 100 μL of 50% solution of acetic acid in 2000 mL of | | |
| | acetonitrile | | |
| Gradient | Time (min) | % Eluent A | % Eluent B |
| | 0 | 60 | 40 |
| | 25 | 60 | 40 |
| | 35 | 55 | 45 |
| | 45 | 30 | 70 |
| | 47 | 10 | 90 |
| | 47.1 | 60 | 40 |
| | 50 | 60 | 40 |

| | |
|---|---|
| Run time | 50 minutes |
| Flow Rate: | 2.3 mL/mins. |
| Detector: | UV at 205 nm |
| Column | 60° C. |
| temperature: | |
| Injection | 20 μl |
| Diluent | acetonitrile |

Retention time:

| | | |
|---|---|---|
| Ascomycin | 29 | min |
| Des-methyl ascomycin (FK-523) RRt | 0.70 | min |
| DL = 0.025% | | |

### HPLC Method for Analyzing Pimecrolimus:

| Column & | Octadecyl silane (OSD or C18) chemically bonded to | | |
|---|---|---|---|
| Packing: | porous silica particles; 150 × 4.6 mm, 3.5 μm | | |
| Eluent A: | 0.02M KH₂PO₄ pH = 4.80 ± 0.05/1 M NaOH): acetonitrile | | |
| | 75:25 mixture | | |
| Eluent B: | 0.02M KH₂PO₄ pH = 4.80 ± 0.05/1 M NaOH): acetonitrile | | |
| | 20:80 mixture | | |
| Gradient | Time (min) | % Eluent A | % Eluent B |
| | 0 | 36 | 64 |
| | 40 | 36 | 64 |
| | 53 | 0 | 100 |
| | 58 | 0 | 100 |
| | 58.1 | 36 | 64 |
| | 64 | 36 | 64 |

| | |
|---|---|
| Run time | 64 minutes |
| Flow Rate: | 0.8 mL/mins. |
| Detector: | UV at 210 nm |
| Column | 55° C. |
| temperature: | |
| Injection | 10 μl |
| Diluent | acetonitrile |

Retention time:

| | | |
|---|---|---|
| Ascomycin | 9.2 | min |
| Pimecrolimus | 37 | min |
| 32-deoxy-32-epichloro-FK523 | 28.5 | min |

### DL=0.02%, QL=0.05%.

The following non-limiting examples are merely illustrative of the preferred embodiments of the present invention, and are not to be construed as limiting the invention.

### Example 1

### Preparation of Preliminary Purified Ascomycin

General description: Ascomycin starting material (crude product) was purified by chromatography and several crystallization steps. The starting material contained 2.03 area percent of des-methylascomycin (FK-523) and 0.96 area percent of impurity RRT: 1.31. An assay of the starting substance gave a purity of 86.8 percent by mass. Purification of the crude ascomycin as described herein produced an ascomycin product that contained 0.36 area percent des-methylascomycin, 0.18 area percent of impurity RRT: 1.31, and 0.094 area percent of impurity RRT: 1.27. The amount of any other impurity was not more than 0.09 area percent, and the HPLC purity of the ascomycin obtained with the method of the invention was 99.2 area percent.

### Chromatography Step of Purification Method

AMBERLITE® XAD 1180 sorption resin was used for chromatographic purification of the crude ascomycin. Two chromatography columns (40 cm diameter, 1 m column height, and ca. 100 liters wet sorption resin) were prepared. The crude ascomycin starting material in an amount of 4000 g, where 3472 g was active substance was dissolved in 30 liters of acetone to produce an ascomycin solution. The resin AMBERLITE® XAD 1180 in an amount of 33 liters was added to the ascomycin solution to produce an ascomycin solution-resin mixture. Water in an amount of 180 liters was slowly added, with agitation, to the ascomycin solution-resin mixture. When the addition of water was complete, the loading charge of sorption resin was collected by filtration.

The collected loading charge was loaded as a layer on the top of the bed of wet sorption resin. The total resin volume was circa 100 liters. The column was first eluted with circa 700 liters of tetrahydrofuran/water (34 vol % THF). A second column was connected to the first column. The elution was carried out with circa 2100 liters of a THF/water mixture (34 vol % THF). The first column was disconnected from the second column, and the elution was continued with circa 1100 liters of eluent of THF/water (34 vol % THF). Fractions having a volume of 20 liters each were collected. Fractions each having a volume of 20 liters were collected and several fractions were analyzed by HPLC.

Appropriate fractions were then combined. However, it should be noted that, prior to the combination of the fractions, preliminary fractions may be combined, e.g., 10 ml from each appropriate fraction, and analyzed with HPLC analysis.

The combined main fraction (circa. 1100 liters) was mixed with 100 ml of 85 percent phosphoric acid, and concentrated at reduced pressure to a volume of about 200 liters. The concentrate was cooled to ambient temperature, and 50 liters of water, 100 liters of ethyl acetate, and 200 ml of concentrated ammonia solution were added to the concentrate. The ethyl acetate phase (circa. 75 liters) was separated, and concentrated under reduced pressure to give an oily residue of ascomycin having appr. 0.4% Of FK-523.

The oily residue was diluted with 10 liters of ethyl acetate, and concentrated again to an oily residue under reduced pressure. The heating temperature was circa 60° C., and the estimated boiling temperature was 20-40° C. The dilution-concentration step was repeated twice.

The solid content of oily residue was established by evaporation of a small amount of sample under reduced pressure, resulting in a solids content of 2476 g for the oily residue. The oily residue was diluted with ethyl acetate to circa 5818 g, and 22.28 1 cyclohexane was added to the solution. The temperature was maintained at 25° C. using a temperature circulator.

Water was added rapidly to the solution in an amount of 198.1 ml. Water in an amount of 346.6 ml was added to the solution for 3 hours, initiating crystallization. After stirring for 90 minutes, the crystals were filtered, and washed with 2476 ml of cyclohexane. The washed crystals were dried at 70° C. for 12 hours, providing a mass of dried crystals of 2027 g

### Recrystallization of Ascomycin

Ascomycin an amount of 2000 g was dissolved in 20 liters of ethyl acetate. The solution was concentrated to an oily residue under reduced pressure. The dissolution-concentration step was repeated. The oily residue (3270 g) was dissolved in 1589 ml ethyl acetate. Cyclohexane in an amount of 18.0 liters was added to the ascomycin solution. Water in an amount of 44 ml was added to the solution for 3 hours, initiating crystallization. After 1.5-2 hour stirring at 20-25° C. the crystals were filtered, and suspended with 6 liters of cyclohexane.

Drying was carried out under reduced pressure at 70° C. for 16 hours. A nitrogen inlet was used during the whole drying process.

The mass of the recrystallized product was 1735.8 g. The HPLC purity was 99.2 area percent, demethyl ascomycin (FK-523) content was 0.36 area percent, didhydrotacrolimus RRT: 1.31 content was 0.18 area percent, and impurity RRT: 1.27 content was 0.094, amount of any other impurity was not more than 0.09 area percent.

### Example 2

### Process for Preparing the Ascomycin Having Less then 0.36% pf Fk-523

General description: The ascomycin starting material (crude product) was purified by chromatography and several crystallization steps, according to the steps described below. The starting material contained 2.03 area percent of des-methylascomycin and 0.96 area percent of impurity RRT: 1.31. An assay of the starting substance gave a purity of 86.8 percent by mass. Following purification according to the present method the product contained 0.12 area percent demethylascomycin, 0.23 area percent of impurity RRT: 1.31, and 0.08 area percent of impurity RRT: 1.1. The amount of any other impurity present was not more than 0.04 area percent, and the purity of the ascomycin obtained with the method of the invention was 99.50 area percent.

### Chromatography Step of Purification Method

AMBERLITE® XAD 1180 sorption resin was used for chromatographic purification. Two chromatography columns (40 cm diameter, 1 m column height, and ca. 100 liters wet sorption resin) were prepared. The crude ascomycin starting material in an amount of 4000 g, where 3472 g was active substance, was dissolved in 30 liters of acetone to produce an ascomycin solution. The resin AMBERLITE® XAD 1180 in an amount of 33 liters was added to the ascomycin solution. Water in an amount of 180 liters was slowly added, with agitation to the ascomycin solution: resin mixture. When the addition of water was complete, the loading charge of sorption resin was collected by filtration.

The collected loading charge was loaded as a layer on the top of the bed of wet sorption resin. The total resin volume was circa 100 liters. The column was first eluted with circa 700 liters of eluent of tetrahydrofuran/water (34 vol % THF). After the first elution, a second column was connected to the first column. The elution was continued with circa 1400 liters of eluent of THF/water (34 vol % THF). The first column was disconnected from the second column, and the elution was continued with circa. 1000 liters of eluent of THF/water (34 vol % THF). Fractions having a volume of 20 liters each were collected. Fractions, each having a volume of 20 liters, were collected and several fractions were analyzed by HPLC.

Appropriate fractions were then combined. However, it should be noted that, prior to the combination of the fractions, preliminary fractions may be combined, e.g., 10 ml from each appropriate fraction, and analyzed with HPLC analysis.

The combined main fraction (circa. 1100 liters) was mixed with 100 ml of 85 percent phosphoric acid, and concentrated at reduced pressure to a volume of about 200 liters. The concentrate was cooled to ambient temperature, and 50 liters of water, 100 liters of ethyl acetate, and 200 ml of concentrated ammonia solution were added to the concentrate. The ethyl acetate phase (circa. 75 liters) was separated, and concentrated under reduced pressure to give an oily residue of ascomycin having 0.48 area percent of FK-523.

Crystallization of Main Fraction after Chromatography

The oily residue was diluted with 10 liters of ethyl acetate, and concentrated again to an oily residue under reduced pressure. The heating temperature was circa 60° C., and the estimated boiling temperature was 20-40° C. The dilution-concentration step was repeated twice.

The solid content of oily residue was established by evaporation of a small amount of sample under reduced pressure, resulting in a solids content of 2440 g for the oily residue. The oily residue was diluted with ethyl acetate to circa 5734 g, and 21.96 1 cyclohexane was added to the solution. The temperature was maintained at 25° C. using a temperature circulator.

Water was added rapidly to the solution in an amount of 195.2 ml. Water in an amount of 341.6 ml was added to the solution for 3 hours, initiating crystallization. After stirring for 90 minutes, the crystals were filtered, and washed with 2440 ml of cyclohexane. The washed crystals were dried at 70° C. for 12 hours, providing a mass of dried crystals of 2030 g, having a purity of 97.2%, and 0.48% area by HPLC of FK-523

### Recrystallization of Ascomycin

Ascomycin an amount of 2000 g was dissolved in 20 liters of ethyl acetate. The solution was concentrated to an oily residue under reduced pressure. The dissolution-concentration step was repeated. The oily residue (2520 g) was dissolved in 2422 ml ethyl acetate. Cyclohexane in an amount of 18 liters was added to the ascomycin solution. Water in an amount of 44 ml was added to the solution for 3 hours, initiating crystallization. After 1.5-2 hour stirring at 20-25° C. the crystals were filtered, and suspended with 6 liters of cyclohexane.

Drying was carried out under reduced pressure at 40° C. for 16 hours. A nitrogen inlet was used during the whole drying process, providing the preliminary purified ascomycin.

The mass of the recrystallized (preliminary purified) product was 1620 g. The HPLC purity was 98.1 area percent, demethylascomycin content (FK-523) was 0.41 area percent, dihydrotacrolimus RRT: 1.31 content was 0.18 area percent.

### Further Recrystallization of the Preliminary Purified Ascomycin.

1st step: Recrystallized ascomycin an amount of 3000 g (combination of 1430 g ascomycin containing demethyl ascomycin: 0.41 area percent, dihydrotacrolimus RRT: 1.31:0.18 area percent and 1570 g ascomycin containing desmethyl ascomycin: 0.38 area percent, impurity RRT: 1.31:0.34 area percent) was dissolved in 10.5 liters of methanol. The temperature was maintained at 60° C. during the crystallization using a temperature circulator. Water in an amount of 7.5 liters was added to the solution for 3 hours, initiating crystallization. After stirring for 2 hours, the crystals were filtered with vacuum, and dried on the filter. 2592 g of air-dried ascomycin was obtained which contained 0.26 area percent desmethyl ascomycin (FK-523), 0.25 area percent of dihydrotacrolimus RRT: 1.31. The HPLC purity of the obtained ascomycin was 99.12 area percent.

2^{nd} step: 1^{st} crystallization step was repeated. The methanol and water amount was calculated to 2592 g starting ascomycin providing 2090 g of air-dried ascomycin was obtained which contained 0.20 area percent demethyl ascomycin, 0.27 area percent of dihydrotacrolimus RRT: 1.31. The HPLC purity of the obtained ascomycin was 99.19 area percent.

3^{rd} step: The air-dried product obtained in the second step an amount of 2090 g was dissolved in 7.3 liters of methanol and the solution was filtered. The temperature was maintained at 60° C. during the crystallization using a temperature circulator. Water in an amount of 4.18 liters was added to the solution for 3 hours, initiating crystallization. After stirring for 2 hours, the crystals were filtered with vacuum, and washed with methanol-water (1:0.7) mixture. Drying was carried out under reduced pressure at 50° C. for 12 hours. A nitrogen inlet was used during the whole drying process. The mass of the final product was 1547 g. The HPLC purity of ascomycin was 99.5 area percent, 0.12 area percent desmethyl ascomycin (FK-523), and 0.23 area percent of dihydrotacrolimus RRT: 1.31, and 0.08 area percent of impurity RRT: 1.1. The amount of any other impurity present was not more than 0.04 area percent, as stated above.

### Example 3

### Preparation of Pimecrolimus from Ascomycin Having Less than 0.36% of FK-523

300 g Ascomycin (prepared in example 1, having 0.036% of FK-523) was dissolved in 1500 ml toluene and concentrated at 40-50° C. The residue was dissolved in 3600 ml toluene-acetonitrile mixture and cooled to −15° C. under dried nitrogen atmosphere. 2100 ml toluene was cooled similarly in another reactor. When the content of the reactors were about −12° C., 150 g trifluoromethanesulfonic anhydride was added to the 2100 ml cold toluene and N,N-diisopropyl-ethylamine (150 ml) was added to the Ascomycin solution. After some minutes stirring, the solution of trifluoromethanesulfonic anhydride was added to the Ascomycin solution by means of overpressure through a PTFE-tube. After 15 minutes, benzyltriethylammonium chloride (360 g) and toluene-acetonitrile mixture (3600 ml) were added to the reaction mixture and it is warmed to 25° C.

The reaction mixture was stirred at this temperature for 1 h, then 1500 ml distilled water was added and after some minutes of vigorous stirring the phases were separated. The lower phase was discarded and fresh water was added (1500 ml). The lower phase was separated again after some minutes of vigorous stirring.

The organic phase was concentrated at 40-50° C. When the organic phase became viscous, toluene was added to it and it was filtered. The filtrate was concentrated further in order to obtain a concentrated solution. Crude pimecrolimus contained 0.34 area percent of 32-deoxy-32-epichloro-FK523.

### Example 4

### Preparation of Pimecrolimus from Ascomycin Having Less than 0.36% of FK-523

Ascomycin that contained 0.12% of FK-523 was subjected to the reaction in example 3 gave pimecrolimus that had 0.11% of 32-deoxy-32-epichloro-FK523.

### Example 5

### Purification of Pimecrolimus

A solution (600 g, approx. 50 w/w %) of crude pimecrolimus (having purity of 78% area containing 0.34% of 32-deoxy-32-epichloro-FK523) obtained from 300 g ascomycin was introduced to a silica column. (4.5 kg silica gel 60, 40-63 μm, eluent: acetone-heptane 1:6) The flask of crude pimecrolimus is washed with 100 g toluene which is also introduced to the column after the 50 w/w %-solution has been soaked into the column.

The pimecrolimus is eluted with acetone-heptane 1:6. The fraction size was 2.5 L, 33 fractions were collected. Fractions #21 to #32 are combined and concentrated at 50° C. to 75% of their original volume. The obtained solution was cooled to 20° C. in 16 h with stirring. After an additional 24 h at 20° C. it was filtered. The yield of solid was 147.5 g. The solid was dissolved in 440 ml acetone and treated with 2200 ml heptane, after stirring overnight at room temperature, the yield was 125.7 g. The solid was then dissolved in 380 ml acetone and treated with 1900 ml of heptane.

After stirring overnight at room temperature the yield was 108.5 g. The purity was determined by HPLC to be 99.39 area % pimecrolimus, and 0.45 Area % 32-deoxy-32-epichloro-FK523. The experiment shows that the both the crude and the purified product have the same amount of impurity: 78.67 area % pimecrolimus and 0.34 area % chlorinated FK-523 vs. 99.39 area % pimecrolimus and 0.45 area % chlorinated FK-523 In normalized area chlorinated FK-523: (100/78.67)×0.34=0.432 vs. (100/99.39)×0.45=0.453

### Example 6

### The Correlation Between the Levels of Fk-523 in Ascomycin and the Level of 32-Deoxy-32-Epichloro-FK523 in PIMECROLIMUS

| FK523 (Area %) | 32-deoxy-32-epichloro-FK523 (Area %) |
|---|---|
| 0.53 | 0.52 |
| 0.74 | 0.83 |
| 0.83 | 0.97 |
| 1.20 | 1.18 |
| 1.65 | 1.53 |

### Example 7

### Crystallization from Hot Methanol

A preliminary purified ascomycin was recrystallized from water:methanol (3.5:1.5) and kept at final crystallization temperature of 50° C. The FK-523 content was reduced from 0.48 area % to 0.275 area %. The yield was 75.6%.

What is claimed is:

1. Ascomycin having less than 0.36% area of FK523 (desmethyl ascomycin).

2. The ascomycin of claim 1, having less than 0.2% area of FK523.

3. The ascomycin of claim 2, having about 0.15% area to about 0.2% area of FK523.

4. Ascomycin that having a purity of at least about 99.2% area.

5. Ascomycin of claim 4, having a purity of at least about 99.5% area.

6. Ascomycin of claim 1, having a purity of at least about 99.2% area.

7. A process for preparing ascomycin comprising providing a preliminary purified ascomycin, and crystallizing the ascomycin from a mixture of an alcohol as a solvent and water as an anti-solvent at a temperature of at least about 45° C.

8. The process of claim 7, wherein the temperature is at least about 60° C.

9. The process of claim 7, wherein crystallization comprises, dissolving the preliminary purified ascomycin in an alcohol and adding water to the solution to obtain a suspension comprising of precipitated ascomycin; wherein the dissolution and precipitation are done at a temperature of at least about 60° C.

10. The process of claim 7 wherein the alcohol is a C₁₋₄ alcohol,

11. The process of claim 10, wherein the alcohol is methanol, ethanol, isopropyl alcohol, n-propyl alcohol, or n-butyl alcohol.

12. The process of claim 11, wherein the alcohol is methanol.

13. The process of claim 7, wherein the addition of water is done over a period of about 10 to about 600 minutes.

14. The process of claim 7, further comprising recovering the ascomycin.

15. The process of claim 7, further comprising repeating the crystallization.

16. The process of claim 7, wherein the obtained ascomycin has a purity of at least about 99.2% area.

17. The process of claim 7, wherein the obtained ascomycin has a purity of at least about 99.5% area.

18. The process of claim 7, wherein the obtained ascomycin has less than 0.36% area of FK523 (desmethyl ascomycin).

19. The process of claim 18, wherein the obtained ascomycin has less than 0.2% area of FK523 (desmethyl ascomycin).

20. The process of claim 19, wherein the obtained ascomycin has about 0.15% area to about 0.2% area of FK523 (desmethyl ascomycin).

21. A process for preparing pimecrolimus comprising converting ascomycin of claim 1 to ascomycin.

22. A process for preparing pimecrolimus, further comprising converting the obtained ascomycin of claim 7 of to pimecrolimus.

23. The process of claim 22, wherein the obtained pimecrolimus has a purity of at least 99.4% area.

24. The process of claim 22, wherein the obtained pimecrolimus has less than 0.45% area of 32-deoxy-32-epichloro-FK523 (desmethyl ascomycin).

25. A process for preparing pimecrolimus having less than 0.45% area of 32-deoxy-32-epichloro-FK523 (desmethyl ascomycin) comprising a) measuring the purity of the ascomycin in at least one batch of ascomycin; b) selecting a batch of ascomycin having less than 0.36% area of FK523, and c) preparing pimecrolimus with less than 0.45% area of 32-deoxy-32-epichloro-FK523 from the selected batch.

26. Pimecrolimus with less than 0.45% area of 32-deoxy-32-epichloro-FK523.

27. Pimecrolimus of claim 26 having a purity of at least 99.4% area.

28. A pharmaceutical composition comprising the Pimecrolimus of claim 26 and at least one pharmaceutically acceptable excipient.

29. A method of treating atopic dermatitis in a human comprising administering the pharmaceutical composition of claim 28 to a human.
